(19) United States
(12) Patent Application Publication
(10) Pub. No.: US 2005/0220733 A1
(43) Pub. Date: Oct. 6, 2005

(54) Skin whitening external preparation

(75) Inventors: Toshi Tsuzuki, Chiba-shi (JP); Eiko Kato, Chiba-shi (JP); Eiji Ogata, Chiba-shi (JP); Hiroshi Ikeno, Itabashi-ku (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K., Tokyo (JP)

(21) Appl. No.: 10/513,371

(22) PCT Filed: May 7, 2003
(86) PCT No.: PCT/JP03/05702

§ 371 Date: Nov. 3, 2004

Related U.S. Application Data
(60) Provisional application No. 60/379,753, filed on May 14, 2002.

(30) Foreign Application Priority Data
May 9, 2002 (JP) 2002-134440

(51) Int. Cl. A61K 7/135

(52) U.S. Cl. 424/62

(57) ABSTRACT

The invention relates to skin whitening external preparations which contain tocopherol phosphate and/or a salt thereof as an active ingredient capable of prevention and/or removal of skin pigmentation, cosmetics including the skin whitening external preparation and cosmetic cares using the cosmetic. The skin whitening external preparations have excellent skin whitening effects and are readily producible so that they can find wide use for the purpose of skin whitening.

### CROSS REFERENCE OF RELATED APPLICATION

This application is an application filed under 35 U.S.C. §111 (a) claiming benefit pursuant to 35 U.S.C. §119 (e) (1) of the filing date of Provisional Application 60/379,753 filed on May 14, 2002 pursuant to 35 U.S.C. §111 (b).

### TECHNICAL FIELD

The present invention relates to skin whitening external preparations which contain tocopherol phosphate and/or a salt thereof as an active ingredient capable of prevention and/or removal of pigmentation, cosmetics comprising the skin whitening external preparation and cosmetic cares using the cosmetic.

### BACKGROUND ART

Many external preparations have been developed for use to treat skin pigmentation, such as spots or freckles by exposure to ultraviolet rays, wounds, burn scars and darkening of surgical scars.

For example, polyphenols are widely known to have depigmentation effects by reducing melanin pigments, and hydroquinones in particular have been clinically used quite often in U.S. and other countries. However, it has been pointed out that the compound causes strong irritation on skin and is hardly a safe drug for skin which can be used without anxiety.

Cosmetics often contain vitamin C or a derivative thereof, such as ascorbic acid 2-phosphate or ascorbic acid 2-glucoside, as a whitening agent. It has been confirmed that oral administration of tocopherols is effective against pigmentation (K. Werininghaus et al., Arch Dermatol, 130, 1257, 1997). Also, effects of suppressed pigmentation by a specific derivative, tocopherol ferulate, have been confirmed with cultured cells (M. Ichihashi et al., Anticancer Res., 19, 3769, 1999). However, there has been no report on successful prevention or removal of pigmentation by percutaneous administration of tocopherol.

Tocopherols knows as vitamin E (e.g., α-tocopherol, β-tocopherol, γ-tocopherol and δ-tocopherol) and derivatives, such as tocopherol acetate and tocopherol nicotinate, have been used in medical drugs, cosmetics and feeds on account of their effects of antioxidation, biomembrane stabilization, immune activation and facilitation of blood circulation.

These tocopherol derivatives are oil soluble to cause difficulties in making preparations, so that water-soluble tocopherol phosphates, amino acid esters and alkylaminocarboxylates have been proposed.

However, there has been no report on anti-pigmentation agents which comprise any of the tocopherols inclusive of the tocopherol phosphates.

### DISCLOSURE OF THE INVENTION

It is an object of the invention to provide highly safe and readily producible skin whitening agents excellent in prevention and/or removal of pigmentation, and particularly cosmetics of excellent whitening ability and cosmetic cares using the cosmetic.

As a result of earnest studies to solve the above problems, the present inventors have found that skin whitening external preparations that comprise tocopherol phosphate and/or a salt thereof have high effects of prevention and removal of pigmentation. The invention has been completed based on the finding.

The invention relates to:

[1] A skin whitening external preparation which comprises tocopherol phosphate and/or a salt thereof as an active ingredient capable of prevention and/or removal of skin pigmentation.

[2] The skin whitening external preparation of [1], wherein the tocopherol phosphate is represented by the formula 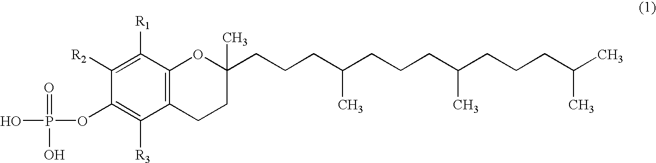
wherein R₁, R₂ and R₃ are each hydrogen or a methyl group.

[3] The skin whitening external preparation of [1] or [2], wherein the tocopherol phosphate is α-tocopherol phosphate.

[4] The skin whitening external preparation of [3], wherein the α-tocopherol phosphate is dl-α-tocopherol phosphate.

[5] The skin whitening external preparation of [3], wherein the α-tocopherol phosphate is d-α-tocopherol phosphate.

[6] The skin whitening external preparation of [1] or [2], wherein the tocopherol phosphate is γ-tocopherol phosphate.

[7] The skin whitening external preparation of [6], wherein the γ-tocopherol phosphate is d-γ-tocopherol phosphate.

[8] The skin whitening external preparation of [1] or [2], wherein the tocopherol phosphate is δ-tocopherol phosphate.

[9] The skin whitening external preparation of [8], wherein the δ-tocopherol phosphate is d-δ-tocopherol phosphate.

[10] The skin whitening external preparation of any of [1] to [9], wherein the salt of tocopherol phosphate is a sodium salt.

[11] The skin whitening external preparation of any of [1] to [10], wherein the content of the tocopherol phosphate and/or the salt thereof is from 0.1 to 10% by mass.

[12] A cosmetic comprising the skin whitening external preparation of any of [1] to [11].

[13] The cosmetic of [12], which is in the form of gel, emulsion or lotion.

[14] A cosmetic care using the cosmetic of [12] or [13].

[15] A use of tocopherol phosphate and/or a salt thereof in skin whitening external preparations as an active ingredient capable of prevention and/or removal of skin pigmentation.

[16] A skin whitening method comprising percutaneously administrating tocopherol phosphate and/or a salt thereof.

### BEST MODE FOR CARRYING OUT THE INVENTION

The effective ingredients, the tocopherol phosphate and the salt thereof, of the invention will be described first.

The tocopherol phosphate is, for example, represented by the formula (1): 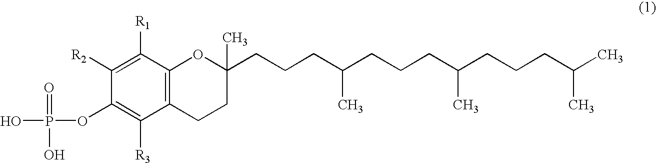
wherein R₁, R₂ and R₃ are each hydrogen or a methyl group.

Preferably, and most usually, the tocopherol phosphate is α-tocopherol phosphate in which R₁, R₂ and R₃ are all methyl groups, γ-tocopherol phosphate in which R₁ and R₂ are both methyl groups and R₃ is hydrogen, or δ-tocopherol phosphate in which R₁ is a methyl group and R₂ and R₃ are both hydrogen.

Examples of preferred salts of the tocopherol phosphate include sodium, potassium, ammonium, alkylammonium, magnesium, calcium and zinc salts.

Of these, sodium salt is particularly preferable since it has high solubility in water and is easy to handle owing to its particulate state.

The tocopherol phosphate of the formula (1) has an asymmetric carbon atom at the second position of the chroman ring, so that the tocopherol phosphate can naturally be a d- or l-stereoisomer or a dl-isomer. The tocopherol phosphate may be any of these isomers.

The tocopherol phosphate and/or the salt thereof can be obtained by a conventional method, e.g., JP-A-59 (1984)/44375 or WO 97/14705.

Specifically, the objective tocopherol phosphate can be obtained by causing a phosphorylating agent, such as phosphorous oxychloride, to react with tocopherol dissolved in a solvent, and appropriately purifying the resulting reaction product.

The objective salt of tocopherol phosphate can be obtained by neutralizing the tocopherol phosphate with a metallic oxide, such as magnesium oxide, a metallic hydroxide, such as sodium hydroxide, ammonium hydroxide or alkylammonium hydroxide.

In the invention, the term “skin whitening” means that the skin whitening external preparations can prevent and/or remove skin pigmentation by the effects of prevention and/or removal of skin pigmentation by the active ingredients tocopherol phosphate and salt thereof.

The active ingredients, the tocopherol phosphate and the salt thereof, capable of prevention and/or removal of skin pigmentation are used in amounts appropriately controlled depending on the objective whitening effects. Preferably, the skin whitening agent contains the tocopherol phosphate and/or the salt thereof in 0.1 to 10% by mass.

For example, the tocopherol phosphate and/or the salt thereof will be desirably contained in 0.1 to 8% by mass in the case of skin whitening lotions, and in 0.1 to 10% by mass in the case of skin whitening gels.

The skin whitening external preparations can be used as cosmetics.

The cosmetic cares of the invention in which the skin whitening external preparation or the cosmetic is used, are also effective for prevention and/or removal of skin pigmentation.

The cosmetics may be any kind of cosmetics usable in contact with skin and can be employed irrespective of age and sex of user. Examples of the cosmetics include skin milks, skin creams, skin foundations, massage creams, cleansing creams, shaving creams, cleansing foams, skin toners, lotions, skin packs, shampoos, conditioners, ointments, bath powders and body soaps.

The skin whitening external preparations may contain other ingredients commonly used in external preparations for skin within limits not detrimental to the effects of the invention. Exemplary ingredients are those listed in The Japanese Standards of Cosmetic Ingredients 2nd edition (edited by Society of Japanese Pharmacopoeia and published by Yakuji Nippo, Ltd. (1984)), The Japanese Cosmetic Ingredients Codex (edited by Ministry of Health and Welfare, Pharmaceutical Examination Division and published by Yakuji Nippo, Ltd. (1993)), Supplement to The Japanese Cosmetic Ingredients Codex (edited by Ministry of Health and Welfare, Pharmaceutical Examination Division and published by Yakuji Nippo, Ltd. (1993)), The Comprehensive Licensing Standards of Cosmetics by Category (edited by Ministry of Health and Welfare, Pharmaceutical Examination Division and published by Yakuji Nippo, Ltd. (1993)) and Dictionary of Cosmetic Ingredients (Nikko Chemicals., Co. Ltd. (1991)). Of these ingredients, the skin whitening preparations can achieve particularly enhanced skin whitening effects by additionally containing a known skin whitening ingredient ascorbic acid or a derivative thereof, such as magnesium ascorbyl 2-phosphate, sodium ascorbyl 2-phosphate, ascorbyl glucoside, sodium ascorbyl 2-phosphoric acid-6-palmitate, sodium ascorbyl 2-phosphoric acid-6-hexyl decanoate, ascorbyl tetraisopalmitate or 3-0-ethyl ascorbate. The above additional ingredients may be contained in amounts within 0.1 to 10%. Other skin whitening ingredients, such as arbutin, ellagic acid, kojic acid, chamomile extract and Rucinol, may by used in combination.

For the purpose of cosmetic care, the cosmetics are preferably in the form of, although not particularly limited to, gel, emulsion or liquid, such as gel emulsions, cosmetic liquids and lotions. Compositions of the emulsions, cosmetic liquids, etc. are not specifically limited and can be in accordance with commonly allied formulations.

### EXAMPLES

The present invention will be described in greater detail by the following Examples, but it should be construed that the invention is in no way limited to those Examples.

In the Examples, the concentrations are in mass percentage.

### Synthetic Example

dl-α-Tocopherol 25.0 g (0.05 mol) was dissolved in toluene 75 ml which contained pyridine 9.3 g. Phosphorous oxychloride 9.8 g (0.064 mol) was dropwise added to the solution with stirring at room temperature (about 15° C.) to 50° C., and reaction was further conducted at room temperature for 3 hours. The precipitated salt was dissolved by addition of 100 ml of a 10% aqueous solution of sulfuric acid, and the organic phase and the aqueous phase were separated. 100 ml of a 10% aqueous solution of sulfuric acid was added to the organic phase, and the mixture was heated under reflux for 4 hours. Then the organic phase was separated, washed with a 5% aqueous solution of sulfuric acid and concentrated to dryness by evaporation to obtain dl-α-tocopherol phosphate.

The dl-α-tocopherol phosphate was dissolved in an alcohol, and the resulting solution was neutralized with a methanol solution of sodium hydroxide. The neutralized solution was added to a methanol-acetone solvent to reprecipitate sodium dl-α-tocopherol phosphate, which was thereafter obtained as white powder.

Corresponding sodium salts of tocopherol phosphate were obtained using d-α-tocopherol, d-γ-tocopherol and d-δ-tocopherol in place of dl-α-tocopherol.

### Example 1

Lotion 1

A homogeneous dispersion-solution consisting of the ingredients 1) to 4) was added to the ingredient 5) with stirring in the following final concentrations, so that an objective lotion was obtained.

| | |
|---|---|
| 1) dl-α-tocopherol phosphate | 2.0 |
| 2) ethanol | 5.0 |
| 3) propylene glycol | 5.0 |
| 4) methyl parahydroxybenzoate | 0.2 |
| 5) purified water | residue |

### Example 2

Lotion 2

A homogeneous dispersion-solution consisting of the ingredients 1) to 4) was added to the ingredient 5) with stirring in the following final concentrations, so that an objective lotion was obtained.

| | |
|---|---|
| 1) sodium dl-α-tocopherol phosphate | 2.0 |
| 2) ethanol | 5.0 |
| 3) propylene glycol | 5.0 |
| 4) methyl parahydroxybenzoate | 0.2 |
| 5) purified water | residue |

### Example 3

Lotion 3

A homogeneous dispersion-solution consisting of the ingredients 1) to 4) was added to the ingredient 5) with stirring in the following final concentrations, so that an objective lotion was obtained.

| | |
|---|---|
| 1) sodium d-α-tocopherol phosphate | 2.0 |
| 2) ethanol | 5.0 |
| 3) propylene glycol | 5.0 |
| 4) methyl parahydroxybenzoate | 0.2 |
| 5) purified water | residue |

### Example 4

Lotion 4

A homogeneous dispersion-solution consisting of the ingredients 1) to 4) was added to the ingredient 5) with stirring in the following final concentrations, so that an objective lotion was obtained.

| | |
|---|---|
| 1) sodium d-γ-tocopherol phosphate | 2.0 |
| 2) ethanol | 5.0 |
| 3) propylene glycol | 5.0 |
| 4) methyl parahydroxybenzoate | 0.2 |
| 5) purified water | residue |

### Example 5

Lotion 5

A homogeneous dispersion-solution consisting of the ingredients 1) to 4) was added to the ingredient 5) with stirring in the following final concentrations, so that an objective lotion was obtained.

| | |
|---|---|
| 1) sodium d-δ-tocopherol phosphate | 2.0 |
| 2) ethanol | 5.0 |
| 3) propylene glycol | 5.0 |
| 4) methyl parahydroxybenzoate | 0.2 |
| 5) purified water | residue |

### Comparative Example 1

Lotion 6

A homogeneous dispersion-solution consisting of the ingredients 1) to 4) was added to the ingredient 5) with stirring in the following final concentrations, so that a lotion containing a commonly used whitening ingredient sodium ascorbyl 2-phosphate, was obtained.

| | |
|---|---|
| 1) sodium ascorbyl 2-phosphate | 2.0 |
| 2) ethanol | 5.0 |
| 3) propylene glycol | 5.0 |
| 4) methyl parahydroxybenzoate | 0.2 |
| 5) purified water | residue |

### Comparative Example 2

Lotion 7

A homogeneous dispersion-solution consisting of the ingredients 1) to 3) was added to the ingredient 4) with stirring in the following final concentrations, so that a lotion containing no whitening ingredient was obtained.

| | |
|---|---|
| 1) ethanol | 5.0 |
| 2) propylene glycol | 5.0 |
| 3) methyl parahydroxybenzoate | 0.2 |
| 4) purified water | residue |

All the lotions obtained as above were homogeneous solutions that had excellent storage stability.

### Example 6

Lotion 8

A homogeneous dispersion-solution consisting of the ingredients 1) to 4) was added to the ingredient 5) with stirring in the following final concentrations, so that an objective lotion was obtained.

| | |
|---|---|
| 1) dl-α-tocopherol phosphate | 0.1 |
| 2) ethanol | 5.0 |
| 3) propylene glycol | 5.0 |
| 4) methyl parahydroxybenzoate | 0.2 |
| 5) purified water | residue |

### Example 7

Lotion 9

A homogeneous dispersion-solution consisting of the ingredients 1) to 4) was added to the ingredient 5) with stirring in the following final concentrations, so that an objective lotion was obtained.

| | |
|---|---|
| 1) sodium dl-α-tocopherol phosphate | 0.1 |
| 2) ethanol | 5.0 |
| 3) propylene glycol | 5.0 |
| 4) methyl parahydroxybenzoate | 0.2 |
| 5) purified water | residue |

### Example 8

Lotion 10

A homogeneous dispersion-solution consisting of the ingredients 1) to 4) was added to the ingredient 5) with stirring in the following final concentrations, so that an objective lotion was obtained.

| | |
|---|---|
| 1) sodium d-α-tocopherol phosphate | 0.1 |
| 2) ethanol | 5.0 |
| 3) propylene glycol | 5.0 |
| 4) methyl parahydroxybenzoate | 0.2 |
| 5) purified water | residue |

### Example 9

Lotion 11

A homogeneous dispersion-solution consisting of the ingredients 1) to 4) was added to the ingredient 5) with stirring in the following final concentrations, so that an objective lotion was obtained.

| | |
|---|---|
| 1) sodium d-γ-tocopherol phosphate | 0.1 |
| 2) ethanol | 5.0 |
| 3) propylene glycol | 5.0 |
| 4) methyl parahydroxybenzoate | 0.2 |
| 5) purified water | residue |

### Example 10

Lotion 12

A homogeneous dispersion-solution consisting of the ingredients 1) to 4) was added to the ingredient 5) with stirring in the following final concentrations, so that an objective lotion was obtained.

| | |
|---|---|
| 1) sodium d-δ-tocopherol phosphate | 0.1 |
| 2) ethanol | 5.0 |
| 3) propylene glycol | 5.0 |
| 4) methyl parahydroxybenzoate | 0.2 |
| 5) purified water | residue |

### Comparative Example 3

Lotion 13

A homogeneous dispersion-solution consisting of the ingredients 1) to 4) was added to the ingredient 5) with stirring in the following final concentrations, so that a lotion containing a commonly used whitening ingredient sodium ascorbyl 2-phosphate, was obtained.

| | |
|---|---|
| 1) sodium ascorbyl 2-phosphate | 0.1 |
| 2) ethanol | 5.0 |
| 3) propylene glycol | 5.0 |
| 4) methyl parahydroxybenzoate | 0.2 |
| 5) purified water | residue |

All the lotions obtained as above were homogeneous solutions that had excellent storage stability.

### Example 11

External gel 1

A homogeneous dispersion of the ingredient 1) in the ingredient 2) was added to the ingredient 3) with stirring in the following final concentrations, so that an objective external gel was obtained.

| | |
|---|---|
| 1) sodium dl-α-tocopherol phosphate | 10 |
| 2) glycerol | 20 |
| 3) octyldodecyl myristate | 70 |

### Example 12

External gel 2

A homogeneous dispersion of the ingredient 1) in the ingredient 2) was added to the ingredient 3) with stirring in the following final concentrations, so that an objective external gel was obtained.

| | |
|---|---|
| 1) sodium d-α-tocopherol phosphate | 10 |
| 2) glycerol | 20 |
| 3) octyldodecyl myristate | 70 |

### Example 13

External gel 3

A homogeneous dispersion of the ingredient 1) in the ingredient 2) was added to the ingredient 3) with stirring in the following final concentrations, so that an objective external gel was obtained.

| | |
|---|---|
| 1) sodium d-γ-tocopherol phosphate | 10 |
| 2) glycerol | 20 |
| 3) octyldodecyl myristate | 70 |

### Example 14

External gel 4

A homogeneous dispersion of the ingredient 1) in the ingredient 2) was added to the ingredient 3) with stirring in the following final concentrations, so that an objective external gel was obtained.

| | |
|---|---|
| 1) sodium d-δ-tocopherol phosphate | 10 |
| 2) glycerol | 20 |
| 3) octyldodecyl myristate | 70 |

### Comparative Example 4

External gel 5

A homogeneous dispersion of the ingredient 1) in the ingredient 2) was added to the ingredient 3) with stirring in the following final concentrations, so that an external gel containing a commonly used whitening ingredient sodium ascorbyl 2-phosphate, was obtained.

| | |
|---|---|
| 1) sodium ascorbyl 2-phosphate | 10 |
| 2) glycerol | 20 |
| 3) octyldodecyl myristate | 70 |

### Comparative Example 5

External gel 6

The ingredient 1) was added to the ingredient 2) with stirring, and the ingredient 3) was added to the mixture, so that an external gel containing no whitening ingredient was obtained.

| | |
|---|---|
| 1) glycerol | 20 |
| 2) octyldodecyl myristate | 70 |
| 3) purified water | 10 |

All the external gels obtained as above were homogeneous dispersions that had excellent storage stability.

(Measurement of Preventive Effects for Pigmentation)

Fifty male guinea pigs (7 weeks old, weiser maple species, SPF) were shorn on their entire backs with electric clippers (0.05 mm blade) and thereafter shaven with an electric shaver. The exposed skin was covered with an adhesive stretch bandage (SILKYTEX, overlaid with aluminum foil on outer surface) which had six windows 1.5 cm×1.5 cm.

Each of the lotions 1-13 and the external gels 1-6 obtained in the Examples 1-14 and the Comparative Examples 1-5, was sequentially applied to 10 window openings in an amount of 0.05 ml per site.

Four hours later, the treated sites were washed with wet absorbent cotton and dried. Then the every guinea pig was secured in a retaining apparatus and exposed to ultraviolet beams (UVB) of medium wavelength by means of an ultraviolet irradiation device (product of Shinano Seisakusho, provided with fluorescent lamps FL 40S/E30 (TOSHIBA LIGHTING & TECHNOLOGY CORPORATION) and six SE lamps), so that the open sites were irradiated with ultraviolet rays in 300 mJ/cm² dose at a distance of about 10 cm.

The irradiation was followed by application of the same lotions 1-13 and the same external gels 1-6 to the same corresponding sites in an amount of 0.05 ml. This procedure was repeatedly carried out for 3 days. After 28 days from the final irradiation, the pigmentation degree was evaluated by marks for each site according to the following criteria. Also, the brightness of skin color was measured with a color difference meter (CR-20 available from MINOLTA Co., Ltd.) at five points: the four corners and the center point of the treated/irradiated site.

Preventive effects for pigmentation were judged based on the average values of the marks (for the 10 sites) and of the brightness (for the 50 points) of the preparation.

Criteria for judgment of pigmentation degree

| | |
|---|---|
| no pigmentation | 0 |
| slight pigmentation | 1 |
| mild pigmentation | 2 |
| moderate pigmentation | 3 |
| severe pigmentation | 4 |

Result (average value)

| Preparation | Average Mark | Brightness |
|---|---|---|
| Lotion 1 | 1.2 | 64.5 |
| Lotion 2 | 1.2 | 64.7 |
| Lotion 3 | 1.0 | 64.7 |
| Lotion 4 | 1.4 | 64.2 |
| Lotion 5 | 1.4 | 64.7 |
| Lotion 6 | 1.4 | 64.6 |
| Lotion 7 | 3.2 | 61.9 |
| Lotion 8 | 2.6 | 63.1 |
| Lotion 9 | 2.4 | 62.8 |
| Lotion 10 | 2.2 | 63.3 |
| Lotion 11 | 2.4 | 62.3 |
| Lotion 12 | 2.6 | 62.6 |
| Lotion 13 | 2.8 | 62.7 |
| External gel 1 | 0.4 | 66.0 |
| External gel 2 | 0.2 | 65.6 |
| External gel 3 | 0.2 | 66.3 |
| External gel 4 | 0.6 | 66.9 |
| External gel 5 | 0.6 | 65.8 |
| External gel 6 | 3.4 | 61.0 |

The above results confirmed excellent pigmentation preventive effects of the present lotions and external gels comparable to those of the common whitening ingredient sodium ascorbyl 2-phosphate.

(Measurement of Removal Effects for Pigmentation)

Fifty male guinea pigs (6 weeks old, weiser maple species, SPF) were shorn on their entire backs with electric clippers (0.05 mm blade) and thereafter shaven with an electric shaver. The exposed skin was covered with an adhesive stretch bandage (SILKYTEX, overlaid with aluminum foil on outer surface) which had six windows 1.5 cm×1.5 cm. Then the every guinea pig was secured in a retaining apparatus and exposed to ultraviolet beams (UVB) of medium wavelength by means of an ultraviolet irradiation device (product of Shinano Seisakusho, provided with fluorescent lamps FL 40S/E30 (TOSHIBA LIGHTING & TECHNOLOGY CORPORATION) and six SE lamps), so that the open sites were irradiated with ultraviolet rays in 750 mJ/cm² dose at a distance of about 10 cm.

Over the period from the 4th day to the 28th day counted from the final irradiation, each of the lotions 1-13 and the external gels 1-6 obtained in the Examples 1-14 and the Comparative Examples 1-5, was sequentially applied to 10 window openings in an amount of 0.05 ml per site twice a day in the morning and the evening.

On the 28th day, the pigmentation degree was evaluated by marks for each site according to the same criteria as in the measurement of preventive effects for pigmentation.

Removal effects for pigmentation were judged based on the average value of the marks (for the 10 sites) of the preparation.

Result (average value)

| Preparation | Average Mark |
|---|---|
| Lotion 1 | 1.8 |
| Lotion 2 | 1.8 |
| Lotion 3 | 1.6 |
| Lotion 4 | 1.8 |
| Lotion 5 | 1.6 |
| Lotion 6 | 1.8 |
| Lotion 7 | 3.4 |
| Lotion 8 | 2.6 |
| Lotion 9 | 2.4 |
| Lotion 10 | 2.2 |
| Lotion 11 | 2.4 |
| Lotion 12 | 2.6 |
| Lotion 13 | 2.6 |
| External gel 1 | 1.6 |
| External gel 2 | 1.8 |
| External gel 3 | 1.6 |
| External gel 4 | 1.6 |
| External gel 5 | 1.4 |
| External gel 6 | 3.6 |

The above results confirmed remarkable pigmentation removal effects of the present lotions and external gels comparable to those of the common whitening ingredient sodium ascorbyl 2-phosphate.

### Example 15

Further, lotions, emulsions and external gels were prepared with the following formulations.

Lotion 14

A homogeneous solution consisting of the ingredients 1) to 4) was added to the ingredient 6) in which the ingredient 5) had been dissolved with stirring in the following final concentrations, so that an objective lotion was obtained.

| | |
|---|---|
| 1) sodium dl-α-tocopherol phosphate | 0.1 |
| 2) ethanol | 5.0 |
| 3) propylene glycol | 5.0 |
| 4) methyl parahydroxybenzoate | 0.2 |
| 5) sodium ascorbyl 2-phosphate | 3.0 |
| 6) purified water | residue |

Lotion 15

A homogeneous solution consisting of the ingredients 1) to 4) was added to the ingredient 6) in which the ingredient 5) had been dissolved with stirring in the following final concentrations, so that an objective lotion was obtained.

| | |
|---|---|
| 1) sodium d-α-tocopherol phosphate | 0.1 |
| 2) ethanol | 5.0 |
| 3) propylene glycol | 5.0 |
| 4) methyl parahydroxybenzoate | 0.2 |
| 5) sodium ascorbyl 2-phosphate | 3.0 |
| 6) purified water | residue |

Lotion 16

A homogeneous solution consisting of the ingredients 1) to 4) was added to the ingredient 6) in which the ingredient 5) had been dissolved with stirring in the following final concentrations, so that an objective lotion was obtained.

| | |
|---|---|
| 1) sodium d-γ-tocopherol phosphate | 0.1 |
| 2) ethanol | 5.0 |
| 3) propylene glycol | 5.0 |
| 4) methyl parahydroxybenzoate | 0.2 |
| 5) sodium ascorbyl 2-phosphate | 3.0 |
| 6) purified water | residue |

Lotion 17

A homogeneous solution consisting of the ingredients 1) to 4) was added to the ingredient 6) in which the ingredient 5) had been dissolved with stirring in the following final concentrations, so that an objective lotion was obtained.

| | |
|---|---|
| 1) sodium dl-α-tocopherol phosphate | 0.1 |
| 2) ethanol | 5.0 |
| 3) propylene glycol | 5.0 |
| 4) methyl parahydroxybenzoate | 0.2 |
| 5) magnesium ascorbyl 2-phosphate | 3.0 |
| 6) purified water | residue |

Lotion 18

A homogeneous solution consisting of the ingredients 1) to 4) was added to the ingredient 6) in which the ingredient 5) had been dissolved with stirring in the following final concentrations, so that an objective lotion was obtained.

| | |
|---|---|
| 1) sodium d-α-tocopherol phosphate | 0.1 |
| 2) ethanol | 5.0 |
| 3) propylene glycol | 5.0 |
| 4) methyl parahydroxybenzoate | 0.2 |
| 5) sodium ascorbyl 2-phosphate | 3.0 |
| 6) purified water | residue |

Lotion 19

A homogeneous solution consisting of the ingredients 1) to 4) was added to the ingredient 6) in which the ingredient 5) had been dissolved with stirring in the following final concentrations, so that an objective lotion was obtained.

| | |
|---|---|
| 1) sodium d-γ-tocopherol phosphate | 0.1 |
| 2) ethanol | 5.0 |
| 3) propylene glycol | 5.0 |
| 4) methyl parahydroxybenzoate | 0.2 |
| 5) magnesium ascorbyl 2-phosphate | 3.0 |
| 6) purified water | residue |

Emulsion 1

A homogeneous solution consisting of the ingredients 1) to 4) was added to the ingredient 6) in which the ingredient 5) had been dissolved with stirring in the following final concentrations, so that an objective emulsion was obtained.

| | |
|---|---|
| 1) sodium dl-α-tocopherol phosphate | 1.0 |
| 2) hydrogenated soybean phospholipid | 10.0 |
| 3) triglyceride 2-ethylhexanoate | 20.0 |
| 4) methyl parahydroxybenzoate | 0.2 |
| 5) sodium ascorbyl 2-phosphate | 3.0 |
| 6) purified water | residue |

Emulsion 2

A homogeneous solution consisting of the ingredients 1) to 4) was added to the ingredient 6) in which the ingredient 5) had been dissolved with stirring in the following final concentrations, so that an objective emulsion was obtained.

| | |
|---|---|
| 1) sodium d-α-tocopherol phosphate | 1.0 |
| 2) hydrogenated soybean phospholipid | 10.0 |
| 3) triglyceride 2-ethylhexanoate | 20.0 |
| 4) methyl parahydroxybenzoate | 0.2 |
| 5) sodium ascorbyl 2-phosphate | 3.0 |
| 6) purified water | residue |

Emulsion 3

A homogeneous solution consisting of the ingredients 1) to 4) was added to the ingredient 6) in which the ingredient 5) had been dissolved with stirring in the following final concentrations, so that an objective emulsion was obtained.

| | |
|---|---|
| 1) sodium d-γ-tocopherol phosphate | 1.0 |
| 2) hydrogenated soybean phospholipid | 10.0 |
| 3) triglyceride 2-ethylhexanoate | 20.0 |
| 4) methyl parahydroxybenzoate | 0.2 |
| 5) sodium ascorbyl 2-phosphate | 3.0 |
| 6) purified water | residue |

Emulsion 4

A homogeneous solution consisting of the ingredients 1) to 4) was added to the ingredient 6) in which the ingredient 5) had been dissolved with stirring in the following final concentrations, so that an objective emulsion was obtained.

| | |
|---|---|
| 1) sodium dl-α-tocopherol phosphate | 1.0 |
| 2) hydrogenated soybean phospholipid | 10.0 |
| 3) triglyceride 2-ethylhexanoate | 20.0 |
| 4) methyl parahydroxybenzoate | 0.2 |
| 5) magnesium ascorbyl 2-phosphate | 3.0 |
| 6) purified water | residue |

Emulsion 5

A homogeneous solution consisting of the ingredients 1) to 4) was added to the ingredient 6) in which the ingredient 5) had been dissolved with stirring in the following final concentrations, so that an objective emulsion was obtained.

| | |
|---|---|
| 1) sodium d-α-tocopherol phosphate | 1.0 |
| 2) hydrogenated soybean phospholipid | 10.0 |
| 3) triglyceride 2-ethylhexanoate | 20.0 |
| 4) methyl parahydroxybenzoate | 0.2 |
| 5) magnesium ascorbyl 2-phosphate | 3.0 |
| 6) purified water | residue |

Emulsion 6

A homogeneous solution consisting of the ingredients 1) to 4) was added to the ingredient 6) in which the ingredient 5) had been dissolved with stirring in the following final concentrations, so that an objective emulsion was obtained.

| | |
|---|---|
| 1) sodium d-γ-tocopherol phosphate | 1.0 |
| 2) hydrogenated soybean phospholipid | 10.0 |
| 3) triglyceride 2-ethylhexanoate | 20.0 |
| 4) methyl parahydroxybenzoate | 0.2 |
| 5) magnesium ascorbyl 2-phosphate | 3.0 |
| 6) purified water | residue |

External Gel 7

A homogeneous solution of the ingredients 1) and 2) in the ingredient 3) was added to the ingredient 4) with stirring in the following final concentrations, so that an objective external gel was obtained.

| | |
|---|---|
| 1) sodium dl-α-tocopherol phosphate | 10.0 |
| 2) sodium ascorbyl 2-phosphate | 10.0 |
| 3) glycerol | 20.0 |
| 4) octyldodecyl myristate | 60.0 |

External Gel 8

A homogeneous solution of the ingredients 1) and 2) in the ingredient 3) was added to the ingredient 4) with stirring in the following final concentrations, so that an objective external gel was obtained.

| | |
|---|---|
| 1) sodium d-α-tocopherol phosphate | 10.0 |
| 2) sodium ascorbyl 2-phosphate | 10.0 |
| 3) glycerol | 20.0 |
| 4) octyldodecyl myristate | 60.0 |

External Gel 9

A homogeneous solution of the ingredients 1) and 2) in the ingredient 3) was added to the ingredient 4) with stirring in the following final concentrations, so that an objective external gel was obtained.

| | |
|---|---|
| 1) sodium d-γ-tocopherol phosphate | 10.0 |
| 2) sodium ascorbyl 2-phosphate | 10.0 |
| 3) glycerol | 20.0 |
| 4) octyldodecyl myristate | 60.0 |

### EFFECT OF THE INVENTION

The skin whitening external preparations contain the tocopherol phosphate and/or the salt thereof as an active ingredient capable of prevention and/or removal of skin pigmentation. The external preparations have excellent skin whitening effects and are readily producible so that they can find wide use for the purpose of skin whitening.

Also, the cosmetics comprising the above skin whitening external preparations may be effectively employed in variety of forms, such as gels, emulsions and liquids, particularly lotions, emulsions, gels and cosmetic liquids.

The cosmetic cares using any of the above skin whitening external preparations and cosmetics are effective for prevention and/or removal of skin pigmentation.

1. A skin whitening external preparation which comprises tocopherol phosphate and/or a salt thereof as an active ingredient capable of prevention and/or removal of skin pigmentation.

2. The skin whitening external preparation of claim 1, wherein the tocopherol phosphate is represented by the formula (1): 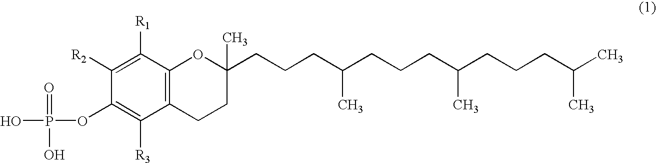
wherein R₁, R₂ and R₃ are each hydrogen or a methyl group.

3. The skin whitening external preparation of claim 1, wherein the tocopherol phosphate is α-tocopherol phosphate.

4. The skin whitening external preparation of claim 3, wherein the α-tocopherol phosphate is dl-α-tocopherol phosphate.

5. The skin whitening external preparation of claim 3, wherein the α-tocopherol phosphate is d-α-tocopherol phosphate.

6. The skin whitening external preparation of claim 2, wherein the tocopherol phosphate is γ-tocopherol phosphate.

7. The skin whitening external preparation of claim 6, wherein the γ-tocopherol phosphate is d-γ-tocopherol phosphate.

8. The skin whitening external preparation of claim 2, wherein the tocopherol phosphate is δ-tocopherol phosphate.

9. The skin whitening external preparation of claim 8, wherein the δ-tocopherol phosphate is d-δ-tocopherol phosphate.

10. The skin whitening external preparation of any of claims 1 to 9, wherein the salt of tocopherol phosphate is a sodium salt.

11. The skin whitening external preparation of any of claims 1 to 9, wherein the content of the tocopherol phosphate and/or the salt thereof is from 0.1 to 10% by mass.

12. A cosmetic comprising the skin whitening external preparation of any of claims 1 to 9.

13. The cosmetic of claim 12, which is in the form of a gel, emulsion or lotion.

14. A cosmetic care using the cosmetic of claim 12.

15. A method of using tocopherol phosphate and/or a salt thereof comprising preparing skin whitening external preparations containing tocopherol phosphate and/or a salt thereof as an active ingredient capable of prevention and/or removal of skin pigmentation.

16. A skin whitening method comprising percutaneously administrating tocopherol phosphate and/or a salt thereof.
